(19) United States
(12) Patent Application Publication
(10) Pub. No.: US 2004/0122382 A1
(43) Pub. Date: Jun. 24, 2004

(54) Elastomeric articles with beneficial coating on a surface

(75) Inventors: David W. Johnson, Alpharetta, GA (US); Alison Bagwell, Cumming, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/328,757

(22) Filed: Dec. 23, 2002

(51) Int. Cl. A61M 35/00

(52) U.S. Cl. 604/292

(57) ABSTRACT

Disclosed is an elastomeric article, such as an elastomeric glove, for example, that includes a coating on the skin-contacting surface of the article. The coating includes a carrier which may separate from the article at expected use conditions and may help to lubricate the skin. The coating also includes an additive which may provide a clinical benefit to the skin. The additive may be an emollient, a humectant, an anti-oxidant, or some other clinically beneficial additive.

### BACKGROUND OF THE INVENTION

Elastomeric materials have been formed into countless different articles suitable for use in many applications, such as surgical gloves, examining gloves, condoms, catheters, balloons, tubing, and the like. Elastomeric materials have been found particularly suitable for such applications due to their physical characteristics. For example, elastomeric materials, in addition to having good elastic properties, exhibit good strength characteristics and may be produced so as to be impermeable not only to aqueous solutions, but also to many solvents and oils.

Elastomeric materials are typically formed so as to be stretched somewhat during normal use. For example, in some elastomeric gloves, the gloves are formed so as to be stretched during donning, in order to fit tightly against the hand and provide good gripping and tactile characteristics during use. In addition, the gloves should be impermeable to substances in order to provide a barrier between the wearer and the environment in which the gloves are used. Unfortunately, the desired characteristics of elastomeric articles may create a harsh environment for the wearer's skin. For example, perspiration is a common problem for glove wearers, and the resulting moist environment may lead to various skin problems, including, for example, growth of fungi and yeast as well as bacterial and viral infections of the skin.

In addition, those who utilize elastomeric articles, such as gloves, are often in clinical conditions that require frequent hand cleaning. For example, clinical personnel must wash their hands or at least wipe their hands with sanitary alcohol formulations many times a day. This constant cleaning may be harsh on the skin, causing excessive skin dryness that may exacerbate skin problems.

In the past, the skin-contacting surface of elastomeric articles was treated with a powder, such as talc or calcium carbonate powder, to absorb some of the moisture and alleviate some of the problems the glove wearers faced. The powder also acted as a barrier between the surface of the article and the skin to make the elastomeric article easier to don. While powder on the article surface is still acceptable for some applications, powders may not be desired in certain applications, such as surgical or other clean-room type applications.

Other methods for treating the skin-contacting surface of elastomeric articles have also been developed. For example, it has been known to coat the inside of a glove with Aloe vera.

Aloe vera is a natural plant extract that has a long history of folk medicine usage. For instance, Aloe vera has been used for external treatment of wounds, burns, and skin irritations, as well as for internal treatment of various conditions. Aloe vera is a popular ingredient in skin-care products. However, research on the clinical benefits of Aloe vera, specifically Aloe vera gel, has been quite contradictory, with trial results varying between no efficacy at all in skin treatment using Aloe vera to ‘miracle’ cures of radiation burns due to treatment with Aloe vera. See, for example, “The Aloe vera Phenomenon: A review of the Properties and Modern Uses of the Leaf Parenchyma Gel” by Douglas Grindlay and T. Reynolds, Journal of Ethnopharmacology, 16(1986)117-161. According to Grindlay and Reynolds, research has shown that Aloe vera is capable of cooling, soothing, and decreasing the pain of burned skin, but no one has apparently succeeded in isolating a single definitive active compound from Aloe vera gel, and industry lacks major clinical trials demonstrating clinical benefit to epithelial tissue from contact with Aloe vera gel.

Another method used in the past for treating the surface of elastomeric articles may be found in U.S. Pat. No. 3,896,807 to Buchalter. Buchalter teaches an article having a surface that is impregnated with the oil phase of a cream formulation, which comprises an oily material and one or more emulsifying agents, and may include emollients, dyes, perfumes and/or pharmaceuticals. However, the oil phase must be a dry, non-oily, non-greasy solid at room temperature, and the formulation of the oil phase impregnant may only be obtained by impregnating the article with a non-aqueous homogenous liquid mixture of the oil phase.

What is needed in the art is a coating composition for the skin-contacting surface of an elastomeric article which may deliver clinical benefits to the skin. Moreover, what is needed is a coating composition that may be applied to the surface of the elastomeric article with a simple, cost-effective process.

### SUMMARY OF THE INVENTION

The present invention is generally directed to a coating composition suitable for the skin-contacting surface of an elastomeric article. In one embodiment, the elastomeric article may be a glove. In general, the coating composition comprises a carrier and a clinically beneficial agent. The carrier may include a material which may separate from the surface of the article at the conditions expected at the article surface during use, for example, a waxy material. In one embodiment, the carrier may include behenetrimonium methosulfate.

The clinically beneficial additive of the present invention may either interact directly with epithelial tissue at the cellular level to provide a benefit to the skin. Alternatively, the clinically beneficial additive may interact with components at or near the skin surface to provide a benefit to the skin.

In one embodiment, the elastomeric article to which the coating is applied may include a primary matrix which includes an elastomeric polymer and at least one skin-contacting surface to which the coating is applied. In one embodiment, the elastomeric polymer may be an elastomeric block copolymer.

### BRIEF DESCRIPTION OF THE DRAWINGS

A full and enabling disclosure of the present invention, including the best mode thereof to one of ordinary skill in the art, is set forth more particularly in the remainder of the specification, including reference to the accompanying figures in which:

FIG. 1 is an illustration of glove-shaped formers that may be used in accordance with one embodiment of the present invention;

FIG. 2 is a front view of a glove according to the present invention; and

FIG. 3 is an enlarged cross-sectional view of one embodiment of an elastomeric article of the present invention.

Repeat use of reference characters in the present specification and drawings is intended to represent same or analogous features or elements of the present invention.

### DETAILED DESCRIPTION OF PREFERRED EMBODIMENTS

It is to be understood by one of ordinary skill in the art that the present discussion is a description of exemplary embodiments only, and is not intended as limiting the broader aspects of the present invention, which broader aspects are embodied in the exemplary construction. Moreover, it should be further understood that even though the elastomeric articles referred to in the remainder of this description are generally referred-to as gloves, the present invention is applicable to other elastomeric articles as well, and is not to be limited to gloves.

In one aspect, the present invention is directed to an elastomeric article that includes a coating composition applied to at least a portion of one surface of the article, such as the inside surface of a glove. When the coated surface is in contact with epithelial tissue during use, naturally occurring water at the tissue surface may contact the coating. At expected use temperatures, at least a portion of the coating may emulsify, dissolve, disperse, or otherwise separate from the glove surface, thereby bringing the coating materials into contact with the tissue so that the coating materials may deliver a clinical benefit to the skin.

The coating used in the present invention includes a carrier that may detach from the glove surface at use conditions, but may remain on the surface of the glove during packaging, shipping, etc. For example, in one embodiment, the carrier may emulsify with water at the skin surface due to transepidermal moisture loss or due to incomplete hand drying. The carrier may, alternatively, separate from the glove surface according to other methods as well. For example, in other embodiments the carrier may dissolve or otherwise disperse in the water available between the skin surface and the glove surface and may deliver the beneficial agents to the skin.

In some embodiments of the present invention, the carrier may provide additional benefits to the elastomeric glove. For example, the carrier may serve to moisturize the skin at use conditions. In one embodiment, the carrier may form a lubricious coating on the glove surface and may enhance donnability of the glove.

The carrier may also enhance the benefits of the other additives in the coating. For example, in certain embodiments, the carrier may be cationic in nature, and may be drawn to the skin surface due to electrostatic attraction which may enhance contact between additives in the coating and the epithelial tissue through, for example, increased residence time. In addition, the skin, in its hydrated state, may be more receptive to the beneficial effects of the agents due to the ionic nature of the carrier.

In addition to the carrier, the coating may also contain an agent or additive that may provide a clinical benefit to the tissue. The term ‘provide a clinical benefit’ is herein defined to mean that there is some interaction or reaction between the epithelial tissue and the additive at the cellular level or alternatively between the epithelial tissue and the additive or some environmental agent and the additive at or near the skin surface, such that some specific benefit is provided to the tissue. An additive that interacts or reacts at the cellular level is one that acts upon the epidermis. An additive that interacts or reacts with the epithelial tissue at or near the skin surface is one that acts upon the stratum corneum. An additive may interact or react with either the epidermis or the stratum corneum, and in some instances, may interact or react with both the epidermis and the stratum corneum. For example, the coating may contain emollients, humectants, moisturizers, vitamins, or other materials that may provide a clinical benefit to the tissue. However, Aloe vera, which, while it may cool and relieve pain in burned skin, has not been found to include a single active compound which interacts with either the tissue at the cellular level or with other elements at the skin surface and as such does not provide a clinical benefit to the skin as herein defined.

Other components or additives may also be included in the coating of the present invention that may further improve the elastomeric article. For example, lubricants, such as silicone lubricants, may be included in the coating of the present invention in order to improve the slip characteristics of the article, including, for instance, damp slip characteristics of the article.

The process of the present invention includes depositing a layer of the coating composition on at least a portion of a surface of an elastomeric article. The coating composition may generally be deposited on a surface of the article that will be in contact with epithelial tissue during use. In one embodiment, this surface may be at least a portion of the donning surface (the inside surface) of the glove. The coating composition may then provide a benefit to the wearer of the article.

In another embodiment, the coating composition may be deposited on at least a portion of the gripping surface (the outside surface). For example, in an embodiment where an elastomeric article is formed that may be used internally, as with a catheter or balloon, for example, it may be preferred to deposit the coating only on the outer surface of the article to deliver the beneficial agents to internal epithelial tissue.

In yet another embodiment, more than one surface is coated with the clinically beneficial composition. For example, the coating composition may be deposited on the donning surface of a medical glove to deliver a clinical benefit to the skin of the wearer, and also be deposited on the gripping surface of the glove to deliver a clinical benefit to the epithelial tissue of a patient. Moreover, the coatings on the two sides of the gloves may include different additives and deliver different benefits to the contacting tissue.

Any elastomeric article, such as a glove, may be processed according to the present invention. For example, a glove may be formed from a natural or a synthetic latex or a dissolved elastomeric polymer. For instance, a glove may be formed of a natural rubber, a nitrile rubber, a polyurethane, a neoprene, a homopolymer of a conjugated diene, a copolymer of a least two conjugated dienes, a copolymer of at least one conjugated diene and at least one vinyl monomer, or any other suitable combinations thereof. Moreover, combinations of polymers or copolymers may be in a single layer of an article or in separate layers, such as in a multi-layer article.

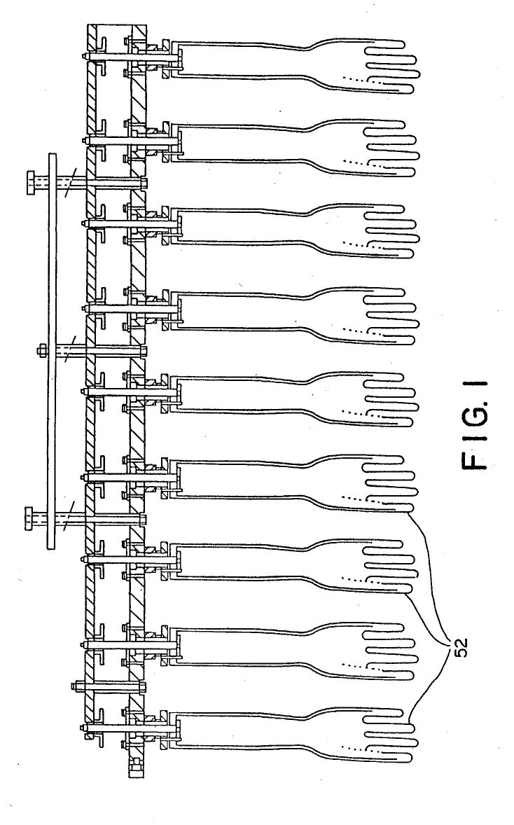

In general, the elastomeric articles of the present invention may be formed by any suitable process. For example, an elastomeric glove may be formed by a series of dipping processes of a former of the shape of the finished article. FIG. 1 is an illustration of a series of glove molds or formers 52 which may be used to form gloves. The formers 52 shown in FIG. 1 are illustrated on a pallet as is conventionally used in a batch processing operation, but it should be understood that a continuous process may alternatively be used. A former 52 may generally be a contoured mold having a textured or smooth surface that may accept a series of coatings and release the formed article. The surface of former 52 may be ceramic, porcelain, glass, metal, or formed from certain fluorocarbons.

If desired, a former may be cleaned prior to formation of a glove on the former. The cleaning process may generally include an optional water pre-rinse followed by an acid wash. After the acid wash, the former may be rinsed with water and dipped in a heated caustic solution prior to a final water rinse. After the cleaning process, a glove may be formed on the former through a series of dipping and drying steps.

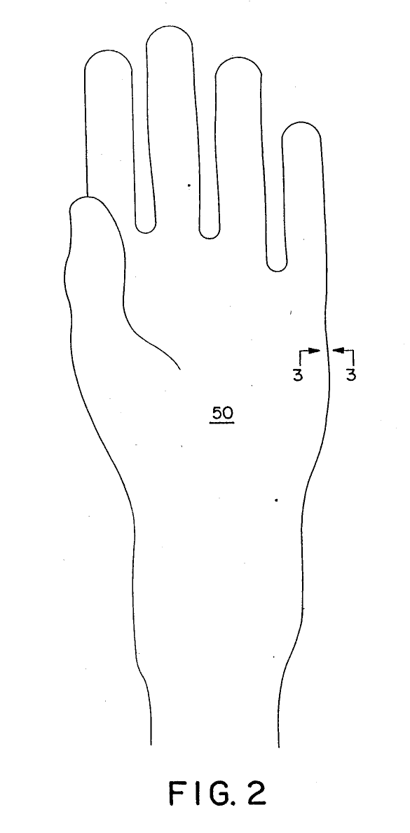

FIG. 2 illustrates one possible embodiment of a glove 50 that may be formed on former 52. In one embodiment, the glove 50 may be formed through a series of dippings or immersions of the former 52. For example, in one embodiment, after cleaning, the former 52 may be dipped into a coagulant composition prior to forming the main body or primary matrix of the glove on the former. For purposes of this disclosure, the primary matrix of the glove is defined to be the main body of the glove and includes one or more layers of elastomeric material. The coagulant causes the base polymer which forms the primary matrix of the glove to coagulate. Coagulant compositions that may be used in the present invention may include powders to ease stripping of the glove from the former, or, if desired, may be powder free. In one embodiment, a powder free coagulant composition may be used which includes water soluble salts of calcium, zinc, aluminum, and the like. For example, calcium nitrate in water or alcohol may be used in the coagulant composition. In such an embodiment, calcium nitrate may be present in the solution in an amount of up to about 40% by weight. The coagulant composition may contain other additives, such as surfactants, that may improve the characteristics of the glove.

After being immersed in the coagulant composition, the former may be withdrawn and the coagulant present on the surface of the former allowed to dry. Once dried, a residual coating of the coagulant is left on the former. The former may then be immersed or dipped into a latex bath of the desired elastomeric polymer. A latex is defined for the purposes of this invention as a colloid in which the elastomeric polymer is suspended in water.

In general, the latex bath of the present invention may have a dry rubber content (DRC) of less than about 50% or alternatively a total solid content (TSC) of less than about 50%. In one embodiment, the latex bath may have a DRC or a TSC content of less than about 25%. The latex bath may also contain various additives such as pH adjustors, stabilizers, and the like.

Upon contact of the latex with the coagulant composition, the coagulant causes some of the latex to become locally unstable and coagulate on the surface of the former. Any additives in the coagulant composition may, depending upon what they are, form a layer between the former and the latex film, or alternatively may be incorporated into the latex film and may subsequently be removed during a leaching process. After the desired amount of time, the former is withdrawn from the latex bath, and the coagulated layer is allowed to coalesce fully on the former.

The amount of time the former is immersed in the emulsion (commonly termed “dwell time”) determines the thickness of the film. Increasing the dwell time of the former in the latex causes the thickness of the film to increase. The total thickness of the film forming the primary matrix may depend on other parameters, including, for example, the solids content of the latex emulsion and the additive content of the latex emulsion and/or the coagulant composition.

In other embodiments, the elastomeric article may be formed from one or more polymers that have been dissolved in a suitable solvent and then allowed to dry on a former in the desired shape. For example, one or more elastomeric block copolymers as are generally known in the art may be dissolved in a solvent, such as toluene, and may then be dried or cured on a former in the shape of the desired elastomeric article. Suitable block copolymers include, for example, styrene-isoprene-styrene (S—I—S) block copolymers, styrene-polybutadiene-styrene (S—B—S) block copolymers, styrene-butadiene (S—B) block copolymers, styrene-ethylene butylene-styrene (S—EB—S) block copolymers, and mixtures thereof.

After formation of the first elastomeric polymer layer, the former may then heated to gel the polymer. The former may then be rinsed in order to leach residual chemicals from the gelled polymer.

Where desired, additional polymeric layers may be formed on the first layer, such that the primary matrix of the glove includes multiple layers. Such a process is generally termed an over-dip process. In one embodiment, an over-dip process may be carried out by immersing the former into an emulsion or a solution of the desired polymer. Additional layers of the primary matrix may enhance certain characteristics of the glove. For instance, an additional layer may provide an improved gripping surface or an improved donning surface on a finished glove. As such, an additional polymeric layer which may improve donning of the glove may be a donning layer, and the coating composition of the present invention may subsequently be deposited on the donning layer.

Following formation of any additional polymeric layers in the primary matrix of the article, a bead rolling operation may be completed.

After the primary matrix of the article is formed including any desired overcoats, the primary matrix of the glove may be finally cured or vulcanized. In general, a natural latex rubber article may be vulcanized at a temperature of between about 80° C. and about 120° C. for from about 10 minutes to about 20 minutes, and a nitrile rubber article may be vulcanized at a temperature of between about 80° C. and about 150° C. for from about 10 minutes to about 20 minutes. In some embodiments, a natural or synthetic rubber latex may be vulcanized by high temperature reaction with a vulcanizing agent, generally sulfur, to cause cross-linking of the polymer chains. In addition to vulcanizing the latex, the high temperature process may cause the evaporation of any volatile components remaining on the former, such as any remaining water, for example. After vulcanization, the glove may be rinsed with water.

In some embodiments, the glove may be stripped from the former and subjected to a halogenation process, such as, for example, chlorination, to improve the surface characteristics of the glove, for example donning slip characteristics. Chlorination may also remove residual proteins and, where a powdered coagulant is used, halogenation may remove residual powder from the surface of the glove The glove may be chlorinated through immersion and optional agitation in an aqueous solution containing dissolved chlorine. In one embodiment, several gloves may be tumbled in a chlorine solution for a period of time between about 10 minutes and about 20 minutes.

After the optional halogenation process, the glove may be rinsed once more in water (preferably soft water) and dried prior to deposition of the presently disclosed coating. For example, a two-step drying process may be utilized in which the gloves are first partially dried by spin-drying in an extractor and then completely dried by being placed in a cyclone dryer.

After drying, the coating composition of the present invention may be applied to at least one surface of the glove. The coating may be applied to at least a portion of the skin-contacting surface of the glove and may contain a carrier and a beneficial agent that may provide a clinical benefit to epithelial tissue, as well as other optional additives.

In general, the carrier is able to separate from the surface of the glove under the temperature and hydration conditions expected during use. Conditions expected during use may be hydration and temperature conditions typical for epithelial tissue, i.e., between about 35° C. and about 40° C. with hydration levels at least equivalent to those due to normal transepidermal moisture loss, though additional moisture may be present due to, for example, water remaining on the skin after washing. Of importance, the coating composition should be formulated so that it will not tend to dissolve, disperse, or otherwise separate from the glove surface prior to use. That is, the coating tends to remain deposited on the glove until it is worn or otherwise in contact with epithelial tissue for a period of time and will not separate from the surface of the glove during packaging, shipping, storage, etc. For example, the carrier may be a self-emulsifying wax, a silicone wax, or another agent which may be compounded so as to prevent softening during packaging and shipment in order to carry the beneficial agent prior to use, and deliver the beneficial agent to the skin surface during use.

The carrier of the coating composition may be cationic, anionic, or nonionic in nature. For example, in certain embodiments, the carrier may be a cationic carrier which may be more substantive to the nature of the skin surface and may, for instance, increase residence time of the coating composition with the skin, thereby potentially enhancing the benefits of the coating composition.

In one embodiment, the carrier may be a self-emulsifying wax. For example, the carrier may be a self-emulsifying wax that includes one or more cationic quaternary ammonium compounds of at least 20 carbon atoms.

In one embodiment, the carrier may include behenetrimonium methosulfate. For example, the carrier may include a quaternary ammonium compound available as a mixture of behenetrimonium methosulfate and cetearyl alcohol under the trade designation Incroquat Behenyl TMS from Croda, Inc. Other possible high molecular weight quaternary ammonium compounds that may be used in the carrier include distearyl dimonium chloride, dimethyl dioctadecyl ammonium chloride, or stearamidopropyl dimethylamine. In another embodiment, the carrier may include stearamidoethyl diethylamine neutralized with hydrochloric acid, citric acid, phosphoric acid, lactic acid, or tartaric acid.

Other exemplary self emulsifying waxes which may be utilized in the present invention include, but are not limited to, Polawax® available from Croda, Inc., which is an emulsifying wax NF; Cosmowax® from Croda, Inc., which is a mixture of cetearyl alcohol and ceteareth 20; Lexemul® 530, which is a glyceryl stearate self-emulsifier; Cosmowax® K from Croda, Inc., which is a mixture of stearyl alcohol and ceteareth 20; Incroquat CR Concentrate, which is a mixture of cetearyl alcohol, PEG-40 castor oil, and stearalkonium chloride; Incroquat BES-35S, which is a mixture of behenamidopropyl ethyldimonium ethosulfate and stearyl alcohol; glyceryl oleate SE; PEG-2 stearate; PEG-2 oleate; PEG-2 laurate; and combinations of suitable self emulsifying waxes.

Other suitable carriers may include mixtures of fatty alcohols, fatty acids, or fatty esters with surfactants having at least 20 carbon atoms. For example, the carrier may include a mixture of fatty acids, fatty alcohols, or fatty esters in combination with a nonionic surfactant, a cationic surfactant, an anionic surfactant, or a mixture of surfactants. In one embodiment, a fatty alcohol may be used including, for example, straight or branched chain, saturated or unsaturated alcohols of at least 12 carbon atoms. For example, lauryl alcohol, myristal alcohol, cetyl alcohol, stearyl alcohol, cetearyl alcohol, or combinations of such alcohols may be used.

One example of a anionic surfactant that may be suitable for use with the present invention is sodium cocoyl isethionate, which is commercially available under the trade designation Geropon® AS-200 from Rhone-Poulenc, Inc.

Other suitable carriers in the present invention may include certain cationic polymers that display the desired transfer characteristics at expected use temperatures. For example, cellulose, collagen, and vinylpyrolidone derived cationic polymers may be used, either individually or in combination.

In one embodiment, cellulose derived polymers may be used such as, for example, Ucare Polymer JR 400 (available from Amerchol, Inc.), or Celquat SC 240 (available from National Starch, Inc.) both of which include the reaction product of hydroxyethyl cellulose and trimethyl ammonium substituted epoxide. Another possible cellulose derived polymer useful in the present invention is Quatrisoft Polymer LM-200 (available from Amerchol, Inc.) and includes the reaction product of polymeric quaternary ammonium salts of hydroxy ethylcellulose and lauryl dimethyl ammonium substituted epoxide.

Other possible cationic polymers that may be used in the carrier include polyquaternium proteins such as, for example, Quat-Coll IP-10 (available from Brooks Industries), polyquaternium-11, and polyquaternium-28 (both of which are available under the trade designation Gafquat from International Specialty Polymers). Mixtures of polyquaternium proteins may also be utilized in the present invention.

The carrier may also include a silicone wax, which may not only carry the clinically beneficial agent and deliver the composition to the skin under use conditions, but may also help to lubricate and soften the skin. Silicone waxes chemically are alkyl polydimethylsiloxanes. A non-limiting list of possible silicone waxes for use in the present invention may include: stearyl dimethicone (Dow Corning 2503), stearyl methicone Clariant SilCare 41M30), C20-C24 alkyl dimethicone (Clariant SilCare 41M70), C20-C24 methicone (Clariant 41M40), C24-C28 dimethicone (Clariant SilCare 41M80), C24-C28 methicone (Clariant SilCare 41M50) and C30-C45 dimethicone (Dow Corning AMS-30).

The coating of the present invention also includes one or more additives that may provide a clinical benefit to the user. In particular, the clinically beneficial additive of the coating may either interact directly with epithelial tissue at the cellular level to provide a benefit to the skin, or alternatively, may interact with components at or near the skin surface in order to provide a benefit to the skin.

In one embodiment, the clinically beneficial additive may be an emollient, which is herein defined as an agent that helps restore dry skin to a more normal moisture balance. Emollients act on the skin by supplying fats and oils that blend in with skin, making it pliable, repairing some of the cracks and fissures in the stratum corneum, and forming a protective film that traps water in the skin. Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

Another possible clinically beneficial additive may include a humectant, which is herein defined to be an agent that supplies the skin with water by attracting moisture from the air and holding it on the skin. Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect your body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; and anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents or extracts that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to the carrier and one or more clinically beneficial additives, other additives may be included in the coating composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino-substituted siloxanes having terminal hydroxy groups, epoxy silicones, quaternary silicones, dimethicone, silicone polyethers, polyether epoxy silicones, silanol fluids, polysiloxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the coating composition may include emulsion stabilizers. Exemplary emulsion stabilizers include aluminum stearate, magnesium sulfate, hydrated silica, and ozokerite.

In one embodiment, a beneficial agent may be held in the coating composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken. The process of forming these beads is called microencapsulation and is generally known in the art.

The coating composition of the present invention may be applied to the gloves as an aqueous solution, a dispersion, an emulsion, or may be applied as an anhydrous composition. In one embodiment, an aqueous composition may be formed including from about 0.1% to about 4% by weight of the carrier and from about 0.001% to about 20% by weight of the clinically beneficial additive, and optionally from about 0.1% to about 4% by weight of a silicone polymer. This composition may then be applied to the surface of an elastomeric article. In one embodiment, an aqueous composition may include from about 0.25% to about 2.5% by weight of the carrier, and from about 0.01% to about 5% by weight of the clinically beneficial additive, and optionally from about 0.25% to about 2.5% by weight of a silicone polymer.

In one embodiment, the coating composition may be applied as an emulsion. In one embodiment, an emulsion may include about a 1% solids content, which may then be applied to the surface of the glove. In one embodiment, the coating composition may be applied to the surface of the glove as a micro-emulsion. A micro-emulsion is a particularly fine-particle emulsion that can be applied in a spray form. The particle size of a micro-emulsion is generally less than about one micron, whereas traditional emulsions demonstrate particle sizes of greater than about 50 microns.

In another embodiment, the composition may be applied as an anhydrous coating. In one embodiment, the anhydrous composition may include from about 80% to about 99% by weight carrier and from about 1% to about 20% by weight beneficial additive. In another embodiment, an anhydrous composition may include from about 80% to about 90% by weight carrier, optionally from about 10% to about 20% by weight silicone polymer, and from about 1% to about 3% by weight beneficial additive.

The components of the coating composition may be applied in combination or separately to the surface of the article. For example, a 100% carrier composition may be applied, followed by a 100% beneficial additive composition, such that the two (or more) separate applications together form the coating of the glove. In such a manner, layers of additives may be built up on the surface of the glove.

The coating may be deposited on the surface of the gloves by any suitable method. For example, the gloves may remain on the formers and the formers may be dipped in the coating. In an alternative embodiment, the gloves may be stripped from the formers and may be tumbled in the coating. In various embodiments, the coating may be applied to the surface of the article through dipping, immersion, spraying, patting, or any other application method known in the art.

In one embodiment, the coating may be sprayed onto a skin-contacting surface of the glove. For instance, after stripping the gloves from the former and following any post-stripping operations, the gloves may be inverted such that the donning side of the glove is once again exposed, and placed in a tumbling apparatus while a solution of the coating is sprayed on the gloves. After being sprayed, the gloves may be dried, for example, the gloves may be tumbled in a dryer for about two to about five minutes at a temperature of about 70° C. In one embodiment, the spraying process may be repeated. For instance, the spraying process may be repeated up to about twenty times to coat the donning surface of the gloves. In one embodiment, the spraying process may be carried out for a total of between about ten and about twenty times.

After applying the coating composition, the gloves may be finally dried, as in a tumble drier, to ensure that the coating on the gloves is anhydrous. For example, the dried coating may be applied to the gloves in an amount of from about 0.001 to about 5 grams per glove. In one embodiment, the dried coating on the surface of the glove may be in an amount of from about 0.01 to about 1 gram per glove. In another embodiment, the dried coating on the surface of the glove may be in an amount from about 0.01 to about 0.10 grams per glove. In another embodiment, the dried coating on the surface of the glove may be in an amount from about 0.01 to about 0.07 grams per glove. In yet another embodiment, the dried coating may be in an amount of about 0.035 grams per glove.

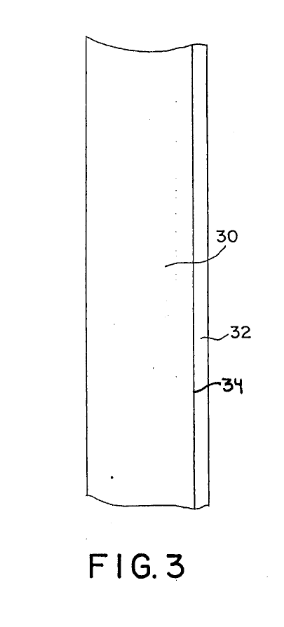

FIG. 3 is an illustration of a cross section of a portion of an article made according to one embodiment of present invention. In this particular embodiment, the primary matrix 30 of the glove is a single layer primary matrix. The coating composition 32 is located at the surface 34 of the primary matrix.

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

What is claimed is:

1. An elastomeric article comprising:
a primary matrix comprising an elastomeric polymer and a skin-contacting surface; and
a coating composition applied to the skin-contacting surface, the coating composition comprising a clinically beneficial additive and a carrier.

2. The elastomeric article of claim 1, wherein the elastomeric polymer comprises a block copolymer.

3. The elastomeric article of claim 2, wherein the block copolymer is selected from the group consisting of styrene-isoprene-styrene block copolymers, styrene-polybutadiene-styrene block copolymers, styrene-butadiene block copolymers, styrene-ethylene butylene-styrene block copolymers, and mixtures thereof.

4. The elastomeric article of claim 1, wherein the carrier comprises a self-emulsifying wax.

5. The elastomeric article of claim 1, wherein the primary matrix is a natural rubber, a nitrile rubber, a polyurethane, or a neoprene.

6. The elastomeric article of claim 1, wherein the carrier comprises a quaternary ammonium compound having at least 20 carbon atoms.

7. The elastomeric article of claim 6, wherein the carrier comprises behenetrimonium methosulfate.

8. The elastomeric article of claim 1, wherein the beneficial additive is selected from the group consisting of an emollient, a humectant, an antioxidant, a neutralizing agent, a chelating agent, an anti-irritant, a vitamin, a skin conditioner, an alpha-hydroxy acid, a moisturizer, a beneficial botanical agent and an extract thereof, and a mixture thereof.

9. The elastomeric article of claim 1, wherein the beneficial additive comprises green tea extract.

10. The elastomeric article of claim 1, wherein the beneficial additive is chamomile or a chamomile extract.

11. The elastomeric article of claim 1, wherein the beneficial additive comprises allantoin.

12. The elastomeric article of claim 1, the coating composition further comprising a silicone polymer.

13. The elastomeric article of claim 12, wherein the silicone polymer comprises dimethicone.

14. The elastomeric article of claim 1, wherein the primary matrix comprises a base layer and a donning layer, wherein the skin-contacting surface of the primary matrix is an exterior surface of the donning layer.

15. The elastomeric article of claim 1, wherein the coating composition is anhydrous on the skin-contacting surface and comprises from about 80% to about 99% by weight carrier and from about 1% to about 20% by weight clinically beneficial additive.

16. The elastomeric article of claim 1, wherein the elastomeric article is a glove.

17. The elastomeric article of claim 16, wherein the coating composition is applied to the glove in an amount from about 0.001 to about 5 grams per glove.

18. The elastomeric article of claim 1, wherein the coating composition does not include Aloe vera.

19. An elastomeric glove comprising:
a primary matrix comprising an elastomeric block copolymer and a skin-contacting surface; and
a coating composition applied to the skin-contacting surface, the coating composition comprising a clinically beneficial additive, a silicone polymer, and a self-emulsifying wax comprising behenetrimonium methosulfate.

20. The elastomeric glove of claim 19, wherein the block copolymer is selected from the group consisting of styrene-isoprene-styrene block copolymers, styrene-polybutadiene-styrene block copolymers, styrene-butadiene block copolymers, styrene-ethylene butylene-styrene block copolymers, and mixtures thereof.

21. The elastomeric glove of claim 19, wherein the clinically beneficial additive is selected from the group consisting of an emollient, a humectant, and a mixture thereof.

22. The elastomeric glove of claim 19, wherein the beneficial additive comprises green tea extract.

23. The elastomeric glove of claim 19, wherein the beneficial additive is chamomile or a chamomile extract.

24. The elastomeric glove of claim 19, wherein the beneficial additive comprises allantoin.

25. The elastomeric glove of claim 19, wherein the silicone polymer comprises dimethicone.

26. The elastomeric glove of claim 19, wherein the primary matrix comprises a base layer and a donning layer, wherein the skin-contacting surface of the primary matrix is an exterior surface of the donning layer.

27. The elastomeric glove of claim 19, wherein the coating composition is an anhydrous composition and comprises from about 80% to about 99% by weight carrier, from about 1% to about 20% by weight clinically beneficial additive, and up to about 20% silicone polymer.

28. The elastomeric glove of claim 19, wherein the coating composition is applied to the glove in an amount from about 0.001 to about 5 grams per glove.

29. The elastomeric glove of claim 19, wherein the composition is applied to the elastomeric article as an aqueous composition, the aqueous composition comprising from about 0.1% to about 4% by weight of the carrier, from about 0.1% to about 4% by weight of the silicone polymer, and from about 0.001% to about 20% by weight of the clinically beneficial additive.
